(19) United States
(12) Patent Application Publication
(10) Pub. No.: US 2026/0120276 A1
(43) Pub. Date: Apr. 30, 2026

(54) INFORMATION PROCESSING DEVICE, INFORMATION PROCESSING METHOD, AND INFORMATION PROCESSING PROGRAM

(71) Applicant: FUJIFILM Corporation, Tokyo (JP)

(72) Inventor: Ryuto MIURA, Tokyo (JP)

(73) Assignee: FUJIFILM Corporation, Tokyo (JP)

(21) Appl. No.: 19/332,091

(22) Filed: Sep. 18, 2025

(30) Foreign Application Priority Data
Sep. 27, 2024 (JP) 2024-169448

(51) Int. Cl. G06T 7/00; A61B 5/00

(52) U.S. Cl. CPC G06T 7/0012; A61B 5/489; G06T 2207/30104; G06T 2207/30172

(57) ABSTRACT

An information processing device including a processor configured to: acquire an input image including a blood vessel; generate course information indicating a blood flow course based on the input image; specify at least a start position of an arterial dissection based on the course information; and discriminate between a true lumen and a false lumen of the arterial dissection based on the course information in a range set in accordance with the start position.

### CROSS-REFERENCE TO RELATED APPLICATIONS

This application claims priority from Japanese Application No. 2024-169448, filed on Sep. 27, 2024, the entire disclosure of which is incorporated herein by reference.

### BACKGROUND

### Technical Field

The present disclosure relates to an information processing device, an information processing method, and an information processing program.

### Related Art

In recent years, curved planar reconstruction (CPR) processing is performed on a three-dimensional image acquired by a computed tomography (CT) apparatus, a magnetic resonance imaging (MRI) apparatus, and the like to improve visibility of an observation part and improve the efficiency of diagnosis. The CPR processing is processing of reconstructing a CPR image which is a two-dimensional image or a three-dimensional image using a randomly set curve direction in the three-dimensional image as one coordinate axis. By using the CPR image, for example, a cross section in a course direction of a blood vessel can be displayed on one screen. For example, JP2013-043016A discloses a technology of creating a linearized CPR image and a volume rendering image from three-dimensional medical image data and displaying the linearized CPR image and the volume rendering image in parallel up and down on a display screen.

In addition, for example, JP2024-505353A discloses a technology of generating a surface mesh of a dissociated blood vessel using a 3D geometric model of the dissociated blood vessel, determining local deformation using the surface mesh, and generating a distortion map of the dissociated blood vessel using a local deformation and the surface mesh.

In recent years, there has been an increasing demand for a technology of enabling support the diagnosis of an arterial dissection. The arterial dissection is a disease in which a fissure forms in the intima of a vascular wall consisting of the intima, media, and adventitia, and blood flows through the fissure into the media. In the arterial dissection, a false lumen is formed due to the dissection in addition to a true lumen, which is an original vascular lumen.

In general, in the diagnosis of the arterial dissection, a treatment plan is made by observing a position and a degree of the arterial dissection, a state of a blood flow in the false lumen, and the like using an image including a blood vessel, such as a CT image. For this purpose, it is required to discriminate between the true lumen and the false lumen from the image, but it may be difficult to discriminate between the true lumen and the false lumen from a local image such as a cross-sectional view perpendicular to the course direction of the blood vessel.

### SUMMARY

The present disclosure provides an information processing device, an information processing method, and an information processing program that can support diagnosis of an arterial dissection.

A first aspect of the present disclosure relates to an information processing device comprising: a processor configured to: acquire an input image including a blood vessel; generate course information indicating a blood flow course based on the input image; specify at least a start position of an arterial dissection based on the course information; and discriminate between a true lumen and a false lumen of the arterial dissection based on the course information in a range set in accordance with the start position.

The processor may be configured to: discriminate between the true lumen and the false lumen based on spatial continuity of the blood flow course indicated by the course information.

The processor may be configured to: discriminate between the true lumen and the false lumen based on a feature value indicating at least one of a shape and a pixel value of the blood vessel indicated by the input image or spatial continuity of the blood flow course indicated by the course information.

The processor may be configured to: further specify an end position of the arterial dissection based on the course information; and discriminate between the true lumen and the false lumen based on the course information in a range set in accordance with the end position in addition to the start position.

The processor may be configured to: determine whether or not the true lumen and the false lumen are continuous at the end position based on the course information; in a case in which it is determined that the true lumen and the false lumen are discontinuous at the end position, discriminate between the true lumen and the false lumen based on the spatial continuity; and in a case in which it is determined that the true lumen and the false lumen are continuous at the end position, discriminate between the true lumen and the false lumen based on the feature value.

The processor may be configured to: estimate a center line of a blood flow region through which blood flows in the blood vessel based on the input image; and generate the course information based on the center line.

The processor may be configured to: in a case in which the center line is interrupted, interpolate the center line based on a distance between the center lines before and after interruption.

The processor may be configured to: acquire a three-dimensional image including the blood vessel as the input image; acquire a coordinate system in a course direction of the blood vessel from the three-dimensional image; generate a curved planar reconstruction (CPR) image along the course direction of the blood vessel based on the coordinate system; and generate the course information based on the CPR image.

The processor may be configured to: acquire a curved planar reconstruction (CPR) image along a course direction of the blood vessel as the input image.

The processor may be configured to: display the start position in the CPR image on a display.

The processor may be configured to: display a region of the true lumen and a region of the false lumen in the CPR image on a display in a distinguishable manner based on a result of discrimination between the true lumen and the false lumen.

The processor may be configured to: receive correction of the result of discrimination; and in a case in which the result of discrimination is corrected, display the region of the true lumen and the region of the false lumen in the CPR image on the display in a distinguishable manner based on the corrected result of discrimination.

The processor may be configured to: display the start position in the input image on a display.

The processor may be configured to: display a region of the true lumen and a region of the false lumen in the input image on a display in a distinguishable manner based on a result of discrimination between the true lumen and the false lumen.

The processor may be configured to: receive correction of the result of discrimination; and in a case in which the result of discrimination is corrected, display the region of the true lumen and the region of the false lumen in the input image on the display in a distinguishable manner based on the corrected result of discrimination.

A second aspect of the present disclosure relates to an information processing method executed by a computer, comprising: acquiring an input image including a blood vessel; generating course information indicating a blood flow course based on the input image; specifying at least a start position of an arterial dissection based on the course information; and discriminating between a true lumen and a false lumen of the arterial dissection based on the course information in a range set in accordance with the start position.

A third aspect of the present disclosure relates to an information processing program causing a computer to execute a process comprising: acquiring an input image including a blood vessel; generating course information indicating a blood flow course based on the input image; specifying at least a start position of an arterial dissection based on the course information; and discriminating between a true lumen and a false lumen of the arterial dissection based on the course information in a range set in accordance with the start position.

According to the above-described aspects, the information processing device, the information processing method, and the information processing program according to the present disclosure can support the diagnosis of the arterial dissection.

### BRIEF DESCRIPTION OF THE DRAWINGS

FIG. 1 is a diagram showing a schematic configuration of an information processing system.

FIG. 2 is a block diagram showing an example of a hardware configuration of an information processing device.

FIG. 3 is a block diagram showing an example of functional configurations of the information processing device.

FIG. 4 is a diagram showing an example of a three-dimensional image.

FIG. 5 is a diagram showing an example of the three-dimensional image.

FIG. 6 is a diagram showing an example of a CPR image.

FIG. 7 is a diagram showing an example of a cross section of the CPR image.

FIG. 8 is a diagram showing course information of Example 1.

FIG. 9 is a diagram showing course information of Example 2.

FIG. 10 is a diagram showing an example of a screen displayed on a display.

FIG. 11 is a flowchart showing an example of information processing.

### DETAILED DESCRIPTION

Hereinafter, an example of an embodiment of the technology of the present disclosure will be described with reference to the drawings. The same or equivalent components and parts in the respective drawings are denoted by the same reference numerals, and duplicated description will be omitted. Furthermore, dimensional ratios in the drawings are exaggerated for convenience of description, and may be different from the actual ratios.

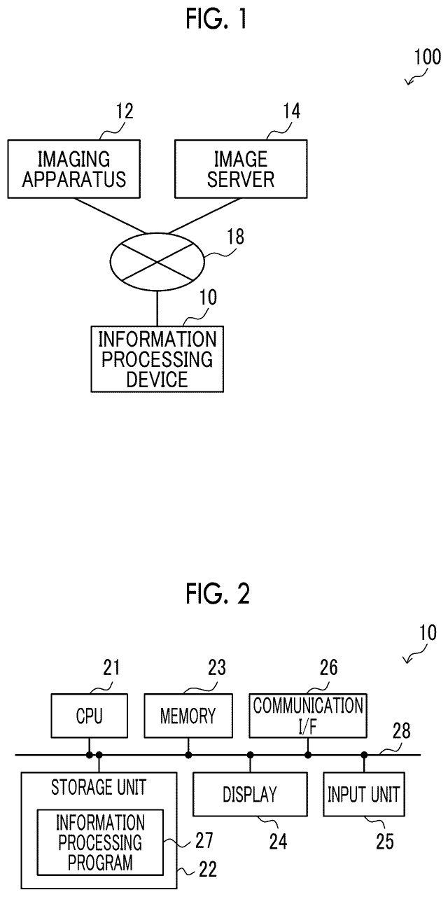

An example of a configuration of an information processing system 100 according to the present embodiment will be described with reference to FIG. 1. The information processing system 100 is a system for supporting a diagnosis of an arterial dissection occurring in an aorta, a coronary artery, a carotid artery, and the like. The arterial dissection is a disease in which the fissure forms in the intima of the vascular wall consisting of the intima, the media, and the adventitia, and blood flows through the fissure into the media. In the arterial dissection, a false lumen is formed due to the dissection in addition to a true lumen, which is an original vascular lumen. In general, in the diagnosis of the arterial dissection, a treatment plan is made by observing a position and a degree of the arterial dissection, a state of a blood flow in the false lumen, and the like using an image including a blood vessel. The information processing system 100 supports the diagnosis of the arterial dissection by discriminating between the true lumen and the false lumen in the arterial dissection.

The information processing system 100 includes an information processing device 10, an imaging apparatus 12, and an image server 14. The information processing device 10, the imaging apparatus 12, and the image server 14 are connected to each other in a communicable state through a wired or wireless network 18. The network 18 is, for example, a local area network (LAN) or a wide area network (WAN).

Each device/apparatus included in the information processing system 100 may be disposed in the same facility (for example, a hospital) or may be disposed in different facilities. In addition, the number of devices/apparatuses included in the information processing system 100 is not particularly limited, and each device/apparatus may be composed of a plurality of devices/apparatuses having the same function.

The imaging apparatus 12 is an apparatus (modality) that generates a medical image T showing a part of a subject, which is to be diagnosed, by imaging the part. The medical image T according to the present embodiment is a three-dimensional image including a blood vessel to be diagnosed. Examples of the imaging apparatus 12 include a computed tomography (CT) apparatus, a magnetic resonance imaging (MRI) apparatus, a positron emission tomography (PET) apparatus, and an ultrasound diagnostic apparatus. The medical image T, which is generated by the imaging apparatus 12, is transmitted to the image server 14.

The image server 14 is a computer that stores and manages various data including the medical image T, and comprises a storage device and database management software. Specifically, the image server 14 acquires the medical image T generated by the imaging apparatus 12 via the network 18, and stores the medical image T in the storage device to manage the medical image T. In a case in which the image server 14 receives an acquisition request for the medical image T from the information processing device 10, the image server 14 transmits the requested medical image T to the information processing device 10. It should be noted that a storage format of various data including the medical image T and communication between the devices/apparatuses are based on a predetermined protocol such as digital imaging and communications in medicine (DICOM).

The information processing device 10 is a device for supporting the diagnosis of the arterial dissection by discriminating between the true lumen and the false lumen in the arterial dissection based on the medical image T acquired by the imaging apparatus 12. Hereinafter, an example of a configuration of the information processing device 10 according to the present embodiment will be described.

An example of a hardware configuration of the information processing device 10 according to the present embodiment will be described with reference to FIG. 2. The information processing device 10 includes a central processing unit (CPU) 21, a non-volatile storage unit 22, and a memory 23 as a temporary storage area. Further, the information processing device 10 includes a display 24, an input unit 25, and a communication interface (I/F) 26. The CPU 21, the storage unit 22, the memory 23, the display 24, the input unit 25, and the communication I/F 26 are connected to each other via a bus 28 such as a system bus and a control bus such that various types of information can be exchanged.

The storage unit 22 is realized by, for example, a storage medium, such as a hard disk drive (HDD), a solid-state drive (SSD), and a flash memory. The storage unit 22 stores an information processing program 27 of the information processing device 10. The CPU 21 reads out the information processing program 27 from the storage unit 22, loads the readout information processing program 27 into the memory 23, and executes the loaded information processing program 27. The CPU 21 is an example of a processor according to the present disclosure.

The display 24 is, for example, a liquid-crystal display, and displays various types of information. The input unit 25 includes a pointing device such as a mouse and a keyboard, and is used to perform various inputs with respect to the information processing device 10. In addition, the display 24 may be configured by a touch panel and used as the input unit 25.

The communication I/F 26 is an interface for communicating with another device/apparatus including the image server 14. For the communication, for example, a wired communication standard, such as Ethernet (registered trademark) or fiber distributed data interface (FDDI), or a wireless communication standard such as 4G, 5G, or Wi-Fi (registered trademark) is used. As the information processing device 10, for example, a server computer, a personal computer, a smartphone, a tablet terminal, a wearable terminal, and the like can be applied as appropriate.

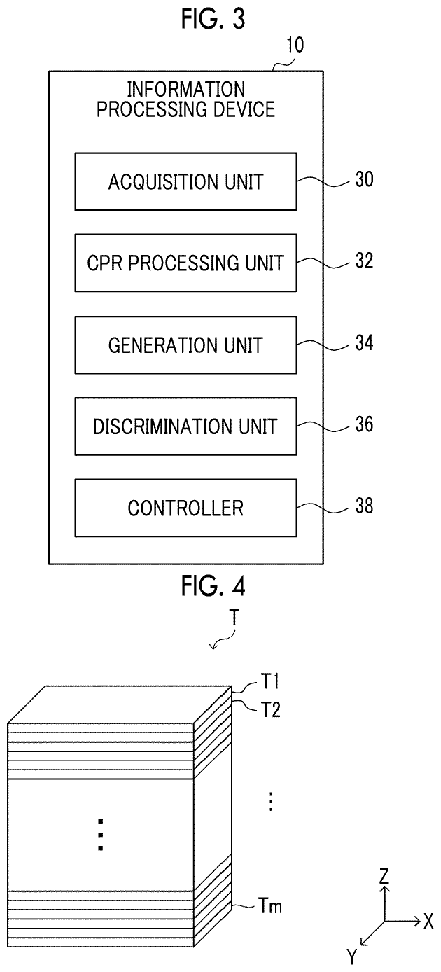

An example of functional configurations of the information processing device 10 according to the present embodiment will be described with reference to FIG. 3. The information processing device 10 includes an acquisition unit 30, a CPR processing unit 32, a generation unit 34, a discrimination unit 36, and a controller 38 as functional configurations. The CPU 21 executes the information processing program 27 to function as the acquisition unit 30, the CPR processing unit 32, the generation unit 34, the discrimination unit 36, and the controller 38.

The acquisition unit 30 acquires an input image including the blood vessel to be diagnosed. Specifically, the acquisition unit 30 acquires a three-dimensional medical image T generated by the imaging apparatus 12 from the image server 14. FIG. 4 schematically shows an example of the medical image T represented in a three-dimensional XYZ coordinate system. The medical image T is, for example, a CT image in which a plurality of tomographic images T1 to Tm (m is 2 or more) representing XY tomographic planes from the head to the waist of one subject (human body) are stacked in a Z direction. The CT image also includes the blood vessel. That is, the acquisition unit 30 acquires the medical image T (three-dimensional image) including the blood vessel as the input image.

The CPR processing unit 32 acquires a coordinate system in a course direction of the blood vessel from the medical image T (three-dimensional image), and generates a curved planar reconstruction (CPR) image along the course direction of the blood vessel based on the coordinate system. The CPR image is a two-dimensional image or a three-dimensional image reconstructed using a randomly set curve direction in the three-dimensional image as one coordinate axis. The “coordinate system in the course direction of the blood vessel” is a two-dimensional or three-dimensional coordinate system including the course direction of the blood vessel as one coordinate axis. That is, the CPR processing unit 32 reconstructs the CPR image based on the medical image T with the course direction of the blood vessel as one coordinate axis.

A known method can be applied as appropriate as a method of generating the CPR image, and an example thereof will be described below. First, the CPR processing unit 32 extracts the blood vessel to be diagnosed from the medical image T (three-dimensional image). As a method of extracting the blood vessel, a known method can be applied as appropriate. For example, the CPR processing unit 32 may extract the blood vessel by performing threshold value processing using a pixel value in the medical image T. Further, for example, the CPR processing unit 32 may extract the blood vessel by template matching using a template representing a shape of the blood vessel. Further, for example, the CPR processing unit 32 may extract the blood vessel using a machine learning model that has been trained in advance to extract the blood vessel from the medical image T.

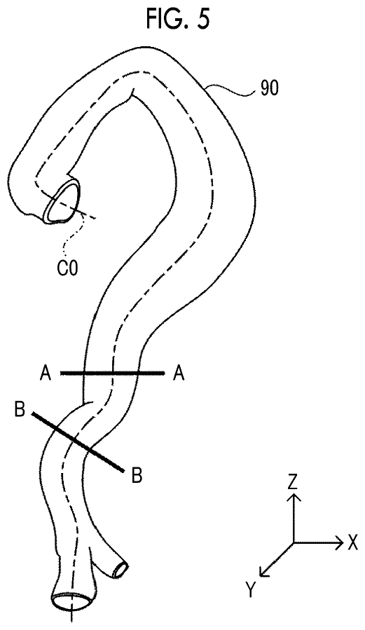

Next, the CPR processing unit 32 sets a core line of the extracted blood vessel. The core line indicates the course direction of the blood vessel and is, for example, a center line of the blood vessel. The core line can be generated, for example, by specifying the center (or centroid) of the blood vessel from each of the tomographic images T1 to Tm and connecting the centers. FIG. 5 shows a blood vessel 90 extracted from the medical image T, and a core line C0 of the blood vessel 90 is indicated by a dashed line.

Next, the CPR processing unit 32 samples cross sections perpendicular to the core line C0 at predetermined intervals (for example, intervals of 1 mm) based on the medical image T (three-dimensional image). FIG. 5 shows an A-A cross section and a B-B cross section as examples of the cross section perpendicular to the core line C0. In the present specification, the term “perpendicular” is not limited to a case in which the two lines intersect at an angle of exactly 90 degrees, and means that the two lines intersect at an angle in a predetermined range including 90 degrees.

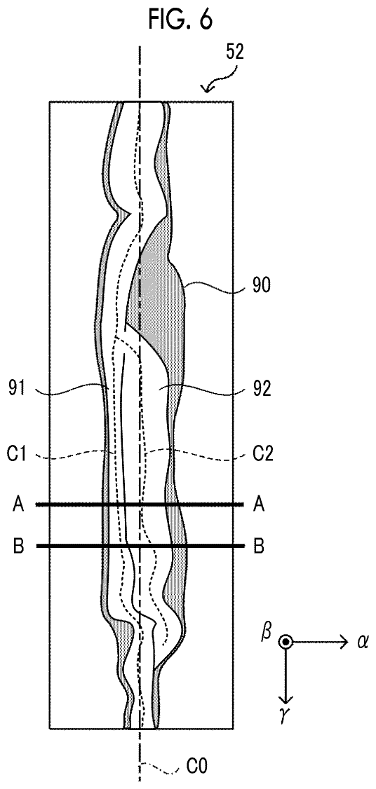

Next, the CPR processing unit 32 stacks the sampled cross section group to generate the CPR image 52 along the course direction of the blood vessel (that is, the direction of the core line C0). FIG. 6 shows a CPR image 52 generated from the blood vessel 90 in FIG. 5. In FIG. 6, the CPR image 52 is represented in an αβγ coordinate system in which the course direction of the blood vessel 90 is defined as a γ direction and a direction perpendicular to the course direction of the blood vessel 90 is defined as an αβ direction. FIG. 6 also shows positions corresponding to the A-A cross section and the B-B cross section in FIG. 5. The CPR processing unit 32 generates the CPR image 52 in which the blood vessel 90 is displayed in a straight line by stacking the cross section group sampled from the medical image T, such as the A-A cross section and the B-B cross section, in the γ direction in parallel with the αβ direction.

The generation unit 34 generates course information indicating a blood flow course based on the input image. Specifically, the generation unit 34 generates the course information based on the CPR image 52 generated by the CPR processing unit 32. Normally, blood flows only through the true lumen, but in a case in which the arterial dissection has occurred, blood also flows through the false lumen. Therefore, the generation unit 34 estimates the blood flow course including the true lumen and the false lumen from the input image, and generates the course information.

Specifically, first, the generation unit 34 extracts a blood flow region in which blood flows in the blood vessel based on the input image (CPR image). In FIG. 6, a first lumen 91 and a second lumen 92 are shown as examples of the blood flow region. For example, the generation unit 34 may extract the blood flow region by performing threshold value processing using a pixel value in the CPR image 52. Further, for example, the generation unit 34 may extract the blood flow region using a machine learning model that has been trained in advance to extract the blood flow region from the CPR image 52.

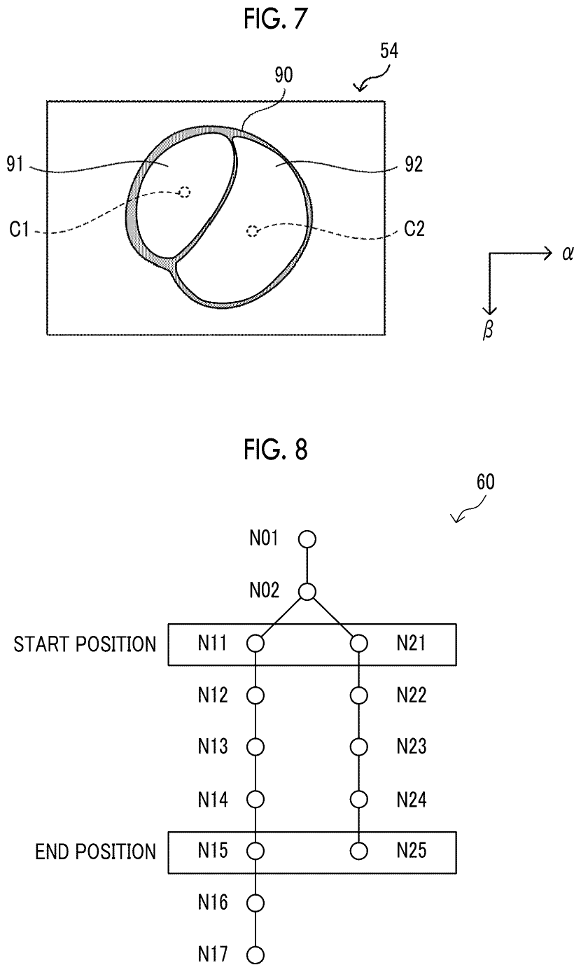

Then, the generation unit 34 estimates the center line of the extracted blood flow region. In FIG. 6, a center line C1 of the first lumen 91 and a center line C2 of the second lumen 92 are each shown by broken lines. The center line can be generated, for example, by specifying the center (or centroid) of each blood flow region from each cross section (for example, the A-A cross section and the B-B cross section) perpendicular to the course direction of the blood vessel 90 in the CPR image 52 and connecting the centers. FIG. 7 shows an example of a certain cross section 54 perpendicular to the course direction of the blood vessel 90 in the CPR image 52. FIG. 7 shows the center line C1 of the first lumen 91 in the cross section 54 and the center line C2 of the second lumen 92 in the cross section 54.

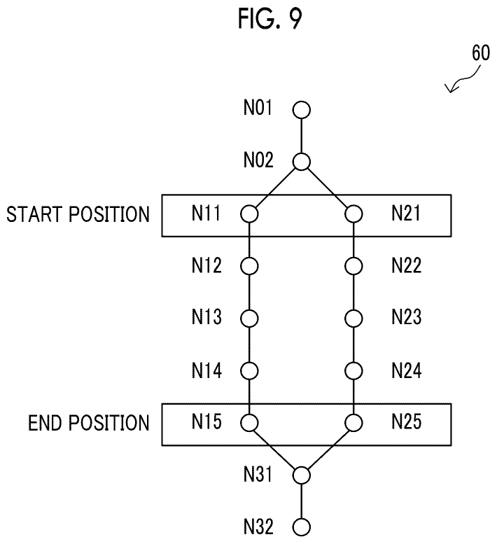

Next, the generation unit 34 generates the course information based on the estimated center line. For example, the generation unit 34 generates a graph represented by a plurality of nodes as the course information by sampling the center line C1 of the first lumen 91 and the center line C2 of the second lumen 92 at predetermined intervals (for example, intervals of 1 mm) (see FIGS. 8 and 9).

In addition, for example, the center line may be interrupted due to a reason such as the blood flow region not being appropriately extracted in some cross sections. In a case in which the center line is interrupted, the generation unit 34 may interpolate the center line based on a distance between the center lines before and after the interruption. For example, in a case in which a plurality of center lines are estimated, the generation unit 34 may estimate that the center lines are interrupted in a case in which the Euclidean distance between the center lines is equal to or less than a predetermined threshold value. In this case, the generation unit 34 may perform linear interpolation on the center line of the interrupted portion and generate the course information (graph) using the center line after the linear interpolation.

The discrimination unit 36 specifies at least a start position of the arterial dissection based on the course information, and discriminates between the true lumen and the false lumen of the arterial dissection based on the course information in a range set in accordance with the start position. Hereinafter, a method of discriminating between the true lumen and the false lumen will be described with reference to examples.

### Example 1

In the present example, the discrimination unit 36 discriminates between the true lumen and the false lumen based on spatial continuity of the blood flow course indicated by the course information. The present example is suitable, for example, in a case in which the true lumen and the false lumen are continuous (entry) at the start position of the arterial dissection, and the true lumen and the false lumen are not continuous at an end position.

FIG. 8 shows an example of a graph 60 generated in such a case. Nodes N11 to N17 are sampled from the center line C1 of the first lumen 91. Nodes N21 to N25 are sampled from the center line C2 of the second lumen 92. Nodes N01 and N02 are sampled from an overlap portion between the center line C1 and the center line C2. Hereinafter, the nodes N11, N21, N15, and N25 corresponding to the start position and the end position of the arterial dissection are referred to as branch nodes.

The discrimination unit 36 specifies the branch nodes N11 and N21 corresponding to the start position of the arterial dissection based on the course information (graph 60). Next, the discrimination unit 36 tracks the connected nodes until any one of the branch nodes N11 or N21 is interrupted. That is, a range from the start position of the arterial dissection to the interruption of the connection of the nodes corresponds to a “range set in accordance with the start position”.

In a case in which the true lumen and the false lumen are not continuous at the end position, it is assumed that the true lumen is longer than the false lumen. Therefore, the discrimination unit 36 determines that the branch node having a longer course (that is, having a larger number of continuous nodes) from each of the branch nodes N11 and N21 is the branch node corresponding to the true lumen.

In the example of FIG. 8, the branch node N11 is continuous with six nodes of the nodes N12 to N17, and the branch node N21 is continuous with four nodes of the nodes N22 to N25. Therefore, the discrimination unit 36 determines that the branch node N11 that is continuous with a larger number of nodes is the branch node N11 corresponding to the true lumen. That is, the discrimination unit 36 determines that the first lumen 91 having the center line C1, which is the sampling source of the branch node N11, is the true lumen and the second lumen 92 is the false lumen.

### Example 2

In the present example, the discrimination unit 36 discriminates between the true lumen and the false lumen based on a feature value indicating at least one of the shape and the pixel value of the blood vessel indicated by the input image (CPR image 52) or the spatial continuity of the blood flow course indicated by the course information (graph 60). In addition, the discrimination unit 36 further specifies the end position of the arterial dissection based on the course information, and discriminates between the true lumen and the false lumen based on the course information in a range set in accordance with the end position in addition to the start position. The present example is suitable, for example, in a case in which the true lumen and the false lumen are continuous (reentry) not only at the start position of the arterial dissection but also at the end position.

FIG. 9 shows an example of the graph 60 generated in such a case. The nodes N11 to N15 are sampled from the center line C1 of the first lumen 91. The nodes N21 to N25 are sampled from the center line C2 of the second lumen 92. The nodes N01 and N02 and the nodes N31 and N32 are sampled from the overlap portion between the center line C1 and the center line C2.

The discrimination unit 36 specifies the branch nodes N11 and N21 corresponding to the start position of the arterial dissection and the branch nodes N15 and N25 corresponding to the end position of the arterial dissection, based on the course information (graph 60). Next, the discrimination unit 36 derives a feature vector at at least one node (node N02) in a predetermined range from the branch nodes N11 and N21 corresponding to the start position. In the same manner, the discrimination unit 36 derives the feature vector at at least one node (node N31) in a predetermined range from the branch nodes N15 and N25 corresponding to the end position. That is, the predetermined range from the start position of the arterial dissection and the predetermined range from the end position correspond to a “range set in accordance with the end position in addition to the start position”.

Even in a case in which the true lumen and the false lumen are continuous (re-entry) at the end position, the true lumen is not always longer than the false lumen, and it is assumed that the true lumen has the same length as the false lumen or is shorter than the false lumen. Therefore, the discrimination unit 36 improves the accuracy of discrimination by using the various feature values described above. Specifically, it is preferable that the discrimination unit 36 uses a multidimensional feature vector including a plurality of various feature values described above. The various feature values or feature vectors can be derived for each point in the vascular lumen, for example, by a feature extractor that receives the CPR image 52 and the graph 60 as input.

The feature vectors have a property that a distance between points included in the same vascular lumen (true lumen or false lumen) is short and a distance between points included in different vascular lumens is long. Therefore, the discrimination unit 36 calculates, for the first lumen 91, a distance between the feature vectors of the branch node N11 and the node N02 corresponding to the start position of the arterial dissection and a distance between the feature vectors of the branch node N15 and the node N31 corresponding to the end position of the arterial dissection. Next, the discrimination unit 36 calculates a score for discrimination between the true lumen and the false lumen using the distance between the feature vectors related to at least one of the start position or the end position of the arterial dissection. For example, the discrimination unit 36 may calculate, as the score, a representative value such as an average value, a total value, a minimum value, and a maximum value of the distances between the feature vectors related to the start position and the end position of the arterial dissection. In addition, for example, the discrimination unit 36 may calculate, as the score, a distance between the feature vectors related to any one of the start position or the end position of the arterial dissection.

Therefore, the discrimination unit 36 calculates, for the second lumen 92, a distance between the feature vectors of the branch node N21 and the node N02 corresponding to the start position of the arterial dissection and a distance between the feature vectors of the branch node N25 and the node N31 corresponding to the end position of the arterial dissection. Then, the discrimination unit 36 calculates the score related to the second lumen 92 in the same manner as the score related to the first lumen 91.

Next, the discrimination unit 36 compares the score related to the first lumen 91 with the score related to the second lumen 92, and determines that the vascular lumen having a smaller score (that is, a closer distance between the feature vectors) is the true lumen. That is, the discrimination unit 36 determines that the vascular lumen having a larger score (that is, a longer distance between the feature vectors) is the false lumen.

The discrimination method using the feature value as in the present example may be applied to an example in which the true lumen and the false lumen are not continuous at the end position, as shown in Example 1. In this case, it may be possible to further improve the accuracy of discrimination.

In addition, for example, whether to use the discrimination method of Example 1 or Example 2 may be switched depending on whether or not the true lumen and the false lumen are continuous at the end position of the arterial dissection. Specifically, the discrimination unit 36 may determine whether or not the true lumen and the false lumen are continuous at the end position based on the course information (graph 60). In a case in which it is determined that the true lumen and the false lumen are discontinuous at the end position as in the example of FIG. 8, the discrimination unit 36 may discriminate between the true lumen and the false lumen based on the spatial continuity as in Example 1. On the other hand, in a case in which it is determined that the true lumen and the false lumen are continuous at the end position as in the example of FIG. 9, the discrimination unit 36 may discriminate between the true lumen and the false lumen based on the feature value as in Example 2.

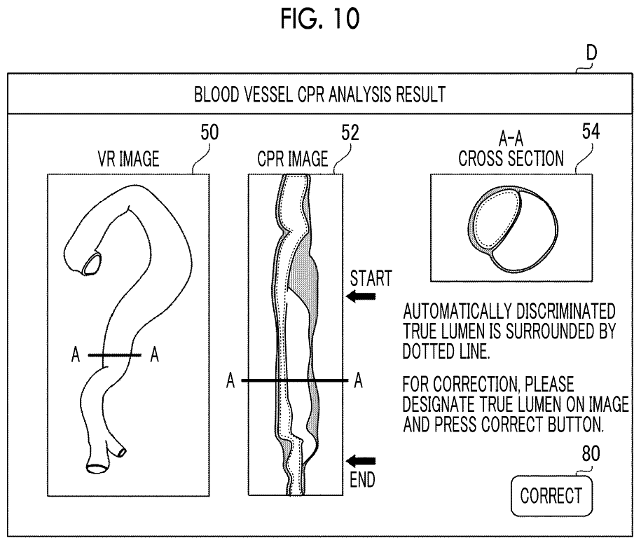

The controller 38 performs control of displaying a result of discrimination between the true lumen and the false lumen on the display 24. FIG. 10 shows an example of a screen D displayed on the display 24 by the controller 38. The screen D includes a volume rendering image 50 (hereinafter, referred to as a VR image 50) of the blood vessel 90 extracted from the CT image that is the input image, the CPR image 52, and the A-A cross section 54.

For example, as shown in FIG. 10, the controller 38 may perform control of displaying a region of the true lumen and a region of the false lumen in the CPR image 52 on the display 24 in a distinguishable manner based on the result of discrimination between the true lumen and the false lumen. In the example of FIG. 10, the region of the true lumen is made distinguishable by surrounding the region of the true lumen with a dotted line. In addition, in the example of FIG. 10, the region of the true lumen is also surrounded by a dotted line in the cross section 54. A method of making the region of the true lumen distinguishable is not particularly limited, and may be, for example, color coding, marking, or display of text such as “The left side of the CPR image is the true lumen”.

In addition, for example, as shown in FIG. 10, the controller 38 may control of displaying at least one of the start position or the end position of the arterial dissection in the CPR image 52 on the display 24. As a result, it is easy for a user to understand a state of the arterial dissection.

In addition, for example, the controller 38 may receive correction of the result of discrimination between the true lumen and the false lumen. In the example of FIG. 10, in a case in which a region different from the region of the true lumen currently being determined on the CPR image 52 or the cross section 54 is designated and a correct button 80 is pressed, the controller 38 changes the region of the true lumen to the designated region. In addition, it is preferable that, in a case in which the result of discrimination is corrected, the controller 38 performs control of displaying the region of the true lumen and the region of the false lumen in the CPR image 52 on the display 24 in a distinguishable manner based on the corrected result of discrimination. That is, it is preferable that the user can check the corrected result of discrimination.

In the example of FIG. 10, the form has been described in which the result of discrimination between the true lumen and the false lumen is displayed using the CPR image 52, but the present disclosure is not limited to this, and the result of discrimination between the true lumen and the false lumen may be displayed using other CT images, the VR image 50, the cross section 54, and the like.

For example, the controller 38 may perform control of displaying at least one of the start position or the end position in the input image on the display 24. In addition, for example, the controller 38 may perform control of displaying the region of the true lumen and the region of the false lumen in the input image on the display 24 in a distinguishable manner based on the result of discrimination between the true lumen and the false lumen. In addition, for example, the controller 38 may receive correction of the result of discrimination between the true lumen and the false lumen, and in a case in which the result of discrimination is corrected, may perform control of displaying the true lumen region and the false lumen region in the input image on the display 24 in a distinguishable manner based on the corrected result of discrimination.

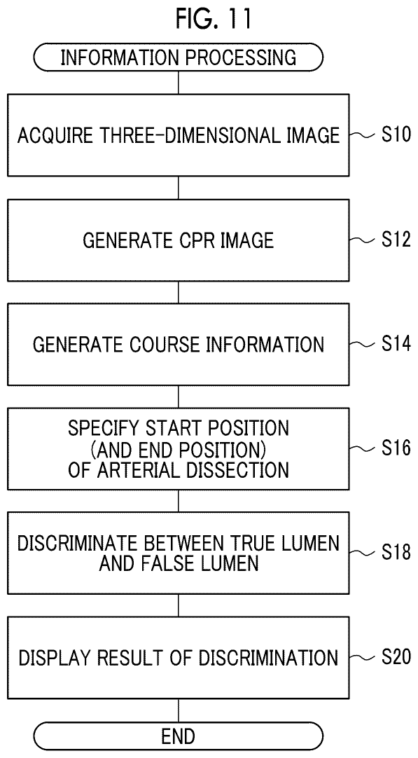

Hereinafter, operations of the information processing device 10 will be described with reference to FIG. 11. In the information processing device 10, the CPU 21 executes the information processing program 27, to execute the information processing shown in FIG. 11. The present processing is executed, for example, in a case in which the user gives an instruction to start the execution.

In step S10, the acquisition unit 30 acquires the three-dimensional image (medical image T), which is generated by the imaging apparatus 12, from the image server 14. In step S12, the CPR processing unit 32 generates the CPR image along the course direction of the blood vessel from the three-dimensional image acquired in step S10. In step S14, the generation unit 34 generates the course information indicating the blood flow course based on the CPR image generated in step S12.

In step S16, the discrimination unit 36 specifies at least the start position of the arterial dissection and, as necessary, the end position based on the course information generated in step S14. In step S18, the discrimination unit 36 discriminates between the true lumen and the false lumen of the arterial dissection based on the course information in the range set in accordance with the start position (and the end position) specified in step S16. In step S20, the controller 38 performs control of displaying the result of discrimination of the true lumen and the false lumen discriminated in step S18 on the display 24, and ends the present information processing.

The information processing device 10 according to the present embodiment comprises the processor as described above. The processor acquires the input image including the blood vessel, generates the course information indicating the blood flow course based on the input image, specifies at least the start position of the arterial dissection based on the course information, and discriminates between the true lumen and the false lumen of the arterial dissection based on the course information in a range set in accordance with the start position.

That is, with the information processing device 10 according to the present embodiment, the true lumen and the false lumen of the arterial dissection can be discriminated from the input image including the blood vessel with high accuracy by specifying the start position of the arterial dissection. Therefore, it is possible to support the diagnosis of the arterial dissection.

In the above-described embodiment, the form has been described in which the CPR processing unit 32 generates the CPR image from the three-dimensional image such as the CT image, but the present disclosure is not limited to this. The CPR processing may be performed by, for example, an external device such as the image server 14. In this case, the acquisition unit 30 acquires the CPR image along the course direction of the blood vessel as the input image from the external device, such as the image server 14, that performs the CPR processing. In this case, the information processing device 10 may omit the function of the CPR processing unit 32.

In addition, in the above-described embodiment, the form has been described in which the input image is a three-dimensional image, but the present disclosure is not limited to this. The input image may be any image as long as the course information of the blood vessel can be generated, and may be, for example, a two-dimensional angiographic image.

In the present embodiment, each processing is executed by any computer. Furthermore, any computer may execute these processes by a processor as hardware, a program as software, or a combination thereof. In such a case, the processor is configured to execute various types of processing in the present embodiment in cooperation with the program, and can function as each unit or each means in the present embodiment. The execution order of the processing by the processor is not limited to the above-described order and may be changed as appropriate. Any computer may be a general-purpose computer, a computer for specific use, a workstation, or another system capable of executing each processing.

The processor may be configured by one or a plurality of types of hardware, and the type of hardware is not limited. The processor may be configured by, for example, a central processing unit (CPU), a micro processing unit (MPU), a programmable logic device such as a field programmable gate array (FPGA), a dedicated circuit for executing specific processing, such as an application specific integrated circuit (ASIC), or hardware such as a graphic processing unit (GPU) or a neural processing unit (NPU). In addition, the types of hardware may be a combination of different types of hardware. In a case in which the plurality of types of hardware are configured to execute one or a plurality of types of processing of a certain processor, the plurality of types of hardware may be present in devices physically separated from each other or may be present in the same device. Further, in any embodiment, the order of each processing performed by the processor is not limited to the above-described order, and may be changed as appropriate. The hardware is configured by an electric circuit (circuitry) in which circuit elements, such as semiconductor elements, are combined, and the like.

Furthermore, the program may be software such as firmware or a microcode. Moreover, the program may be, for example, a program module group, and each function thereof may be implemented by a processor configured to execute each function. The program may be a program code or a plurality of code segments stored in one or a plurality of non-transitory computer-readable media (for example, a storage medium and other storages). The program may be stored in the plurality of non-transitory computer-readable media present in physically separated devices. The program code or the code segment may represent any combination of procedures, functions, subprograms, routines, subroutines, modules, software packages, classes, instructions, data structures, or program statements. The program code or the code segment may be connected to another code segment or a hardware circuit by transmitting and receiving information, data, arguments, parameters, or contents in the memory.

Further, in the above-described embodiment, the aspect has been described in which the information processing program 27 is stored (installed) in the storage unit 22 in advance, but the present disclosure is not limited to this. The information processing program 27 may be provided in a form being recorded in a recording medium such as a compact disc read-only memory (CD-ROM), a digital versatile disc read-only memory (DVD-ROM), and a Universal Serial Bus (USB) memory. Further, a form may be adopted in which the information processing program 27 is downloaded from an external apparatus via the network.

The technology of the present disclosure extends to any program products. The program products include products in any aspect for providing the program. For example, the program product includes a program provided through a network such as the Internet, and a non-transitory computer-readable recording media such as a CD-ROM, a DVD-ROM, and a USB memory in which the program is stored.

In the technology of the present disclosure, the above-described embodiment and modification examples can be combined as appropriate. The above-described contents and the above-shown contents are detailed descriptions of portions related to the technology of the present disclosure and are merely examples of the technology of the present disclosure. For example, the description of the configuration, the function, the operation, and the effect above are the description of examples of the configuration, the function, the operation, and the effect of the parts according to the technology of the present disclosure. Accordingly, it goes without saying that unnecessary parts may be deleted, new elements may be added, or replacements may be made with respect to the above-described contents and the above-shown contents within a range that does not deviate from the gist of the technology of the present disclosure.

The following supplementary notes are disclosed with regard to the above-described embodiment.

### Supplementary Note 1

An information processing device comprising: a processor configured to: acquire an input image including a blood vessel; generate course information indicating a blood flow course based on the input image; specify at least a start position of an arterial dissection based on the course information; and discriminate between a true lumen and a false lumen of the arterial dissection based on the course information in a range set in accordance with the start position.

### Supplementary Note 2

The information processing device according to supplementary note 1, in which the processor is configured to: discriminate between the true lumen and the false lumen based on spatial continuity of the blood flow course indicated by the course information.

### Supplementary Note 3

The information processing device according to supplementary note 1 or 2, in which the processor is configured to: discriminate between the true lumen and the false lumen based on a feature value indicating at least one of a shape and a pixel value of the blood vessel indicated by the input image or spatial continuity of the blood flow course indicated by the course information.

### Supplementary Note 4

The information processing device according to supplementary note 3, in which the processor is configured to: further specify an end position of the arterial dissection based on the course information; and discriminate between the true lumen and the false lumen based on the course information in a range set in accordance with the end position in addition to the start position.

### Supplementary Note 5

The information processing device according to supplementary note 4, in which the processor is configured to: determine whether or not the true lumen and the false lumen are continuous at the end position based on the course information; in a case in which it is determined that the true lumen and the false lumen are discontinuous at the end position, discriminate between the true lumen and the false lumen based on the spatial continuity; and in a case in which it is determined that the true lumen and the false lumen are continuous at the end position, discriminate between the true lumen and the false lumen based on the feature value.

### Supplementary Note 6

The information processing device according to any one of supplementary notes 1 to 5, in which the processor is configured to: estimate a center line of a blood flow region through which blood flows in the blood vessel based on the input image; and generate the course information based on the center line.

### Supplementary Note 7

The information processing device according to supplementary note 6, in which the processor is configured to: in a case in which the center line is interrupted, interpolate the center line based on a distance between the center lines before and after interruption.

### Supplementary Note 8

The information processing device according to any one of supplementary notes 1 to 7, in which the processor is configured to: acquire a three-dimensional image including the blood vessel as the input image; acquire a coordinate system in a course direction of the blood vessel from the three-dimensional image; generate a curved planar reconstruction (CPR) image along the course direction of the blood vessel based on the coordinate system; and generate the course information based on the CPR image.

### Supplementary Note 9

The information processing device according to any one of supplementary notes 1 to 7, in which the processor is configured to: acquire a curved planar reconstruction (CPR) image along a course direction of the blood vessel as the input image.

### Supplementary Note 10

The information processing device according to supplementary note 8 or 9, in which the processor is configured to: display the start position in the CPR image on a display.

### Supplementary Note 11

The information processing device according to any one of supplementary notes 8 to 10, in which the processor is configured to: display a region of the true lumen and a region of the false lumen in the CPR image on a display in a distinguishable manner based on a result of discrimination between the true lumen and the false lumen.

### Supplementary Note 12

The information processing device according to supplementary note 11, in which the processor is configured to: receive correction of the result of discrimination; and in a case in which the result of discrimination is corrected, display the region of the true lumen and the region of the false lumen in the CPR image on the display in a distinguishable manner based on the corrected result of discrimination.

### Supplementary Note 13

The information processing device according to any one of supplementary notes 1 to 12, in which the processor is configured to: display the start position in the input image on a display.

### Supplementary Note 14

The information processing device according to any one of supplementary notes 1 to 13, in which the processor is configured to: display a region of the true lumen and a region of the false lumen in the input image on a display in a distinguishable manner based on a result of discrimination between the true lumen and the false lumen.

### Supplementary Note 15

The information processing device according to supplementary note 14, in which the processor is configured to: receive correction of the result of discrimination; and in a case in which the result of discrimination is corrected, display the region of the true lumen and the region of the false lumen in the input image on the display in a distinguishable manner based on the corrected result of discrimination.

### Supplementary Note 16

An information processing method executed by a computer, comprising: acquiring an input image including a blood vessel; generating course information indicating a blood flow course based on the input image; specifying at least a start position of an arterial dissection based on the course information; and discriminating between a true lumen and a false lumen of the arterial dissection based on the course information in a range set in accordance with the start position.

### Supplementary Note 17

An information processing program causing a computer to execute a process comprising: acquiring an input image including a blood vessel; generating course information indicating a blood flow course based on the input image; specifying at least a start position of an arterial dissection based on the course information; and discriminating between a true lumen and a false lumen of the arterial dissection based on the course information in a range set in accordance with the start position.

What is claimed is:

1. An information processing device comprising a processor configured to:
acquire an input image including a blood vessel;
generate course information indicating a blood flow course based on the input image;
specify at least a start position of an arterial dissection based on the course information; and
discriminate between a true lumen and a false lumen of the arterial dissection based on the course information in a range set in accordance with the start position.

2. The information processing device according to claim 1, wherein the processor is configured to discriminate between the true lumen and the false lumen based on spatial continuity of the blood flow course indicated by the course information.

3. The information processing device according to claim 1, wherein the processor is configured to discriminate between the true lumen and the false lumen based on a feature value indicating at least one of a shape and a pixel value of the blood vessel indicated by the input image or spatial continuity of the blood flow course indicated by the course information.

4. The information processing device according to claim 3, wherein the processor is configured to:
further specify an end position of the arterial dissection based on the course information; and
discriminate between the true lumen and the false lumen based on the course information in a range set in accordance with the end position in addition to the start position.

5. The information processing device according to claim 4, wherein the processor is configured to:
determine whether or not the true lumen and the false lumen are continuous at the end position based on the course information;
in a case in which it is determined that the true lumen and the false lumen are discontinuous at the end position, discriminate between the true lumen and the false lumen based on the spatial continuity; and
in a case in which it is determined that the true lumen and the false lumen are continuous at the end position, discriminate between the true lumen and the false lumen based on the feature value.

6. The information processing device according to claim 1, wherein the processor is configured to:
estimate a center line of a blood flow region through which blood flows in the blood vessel based on the input image; and
generate the course information based on the center line.

7. The information processing device according to claim 6, wherein the processor is configured to in a case in which the center line is interrupted, interpolate the center line based on a distance between the center lines before and after interruption.

8. The information processing device according to claim 1, wherein the processor is configured to:
acquire a three-dimensional image including the blood vessel as the input image;
acquire a coordinate system in a course direction of the blood vessel from the three-dimensional image;
generate a curved planar reconstruction (CPR) image along the course direction of the blood vessel based on the coordinate system; and
generate the course information based on the CPR image.

9. The information processing device according to claim 1, wherein the processor is configured to acquire a curved planar reconstruction (CPR) image along a course direction of the blood vessel as the input image.

10. The information processing device according to claim 8, wherein the processor is configured to display the start position in the CPR image on a display.

11. The information processing device according to claim 8, wherein the processor is configured to display a region of the true lumen and a region of the false lumen in the CPR image on a display in a distinguishable manner based on a result of discrimination between the true lumen and the false lumen.

12. The information processing device according to claim 11, wherein the processor is configured to:
receive correction of the result of discrimination; and
in a case in which the result of discrimination is corrected, display the region of the true lumen and the region of the false lumen in the CPR image on the display in a distinguishable manner based on the corrected result of discrimination.

13. The information processing device according to claim 9, wherein the processor is configured to display the start position in the CPR image on a display.

14. The information processing device according to claim 9, wherein the processor is configured to display a region of the true lumen and a region of the false lumen in the CPR image on a display in a distinguishable manner based on a result of discrimination between the true lumen and the false lumen.

15. The information processing device according to claim 14, wherein the processor is configured to:
receive correction of the result of discrimination; and
in a case in which the result of discrimination is corrected, display the region of the true lumen and the region of the false lumen in the CPR image on the display in a distinguishable manner based on the corrected result of discrimination.

16. The information processing device according to claim 1, wherein the processor is configured to display the start position in the input image on a display.

17. The information processing device according to claim 1, wherein the processor is configured to display a region of the true lumen and a region of the false lumen in the input image on a display in a distinguishable manner based on a result of discrimination between the true lumen and the false lumen.

18. The information processing device according to claim 17, wherein the processor is configured to:
receive correction of the result of discrimination; and
in a case in which the result of discrimination is corrected, display the region of the true lumen and the region of the false lumen in the input image on the display in a distinguishable manner based on the corrected result of discrimination.

19. An information processing method executed by a computer, comprising:
acquiring an input image including a blood vessel;
generating course information indicating a blood flow course based on the input image;
specifying at least a start position of an arterial dissection based on the course information; and
discriminating between a true lumen and a false lumen of the arterial dissection based on the course information in a range set in accordance with the start position.

20. A non-transitory computer-readable storage medium storing an information processing program causing a computer to execute a process comprising:
acquiring an input image including a blood vessel;
generating course information indicating a blood flow course based on the input image;
specifying at least a start position of an arterial dissection based on the course information; and
discriminating between a true lumen and a false lumen of the arterial dissection based on the course information in a range set in accordance with the start position.
